(19) United States
(12) Patent Application Publication
(10) Pub. No.: US 2021/0189505 A1
(43) Pub. Date: Jun. 24, 2021

(54) ASSESSING MICROSATELLITE INSTABILITY BY LIQUID BIOPSY

(71) Applicant: NANTCELL, INC., Culver City, CA (US)

(72) Inventor: Shahrooz Rabizadeh, Culver City, CA (US)

(21) Appl. No.: 17/270,407

(22) PCT Filed: Aug. 22, 2019
(86) PCT No.: PCT/US2019/047646

§ 371 Date: Feb. 22, 2021

Related U.S. Application Data
(60) Provisional application No. 62/722,109, filed on Aug. 23, 2018.

(51) Int. Cl. C12Q 1/6886; C12Q 1/686

(52) U.S. Cl. CPC C12Q 1/6886; C12Q 2600/156; C12Q 2600/106; C12Q 1/686

(57) ABSTRACT

The present disclosure relates generally to methods for detecting and/or treating cancer by assessing circulating free nucleic acids derived from a bodily fluid sample. In particular, in some embodiments, the disclosed methods involve assessing sequence alteration and/or a fragment-size alteration within microsatellite short tandem repeats which are characteristic to certain cancers and some other health conditions.

This application claims priority to U.S. Provisional Application Ser. No. 62/722,109, filed on Aug. 23, 2018. The Provisional Application is incorporated by reference in its entirety.

### FIELD

The present disclosure relates generally to the field of molecular biology and medicine, including methods for detecting and/or treating cancer by assessing circulating free nucleic acids derived from suitable bodily fluid samples such as, e.g., urine, saliva, and blood samples. In particular, in some embodiments, the disclosed methods involve assessing sequence alteration and/or fragment-size alteration within microsatellite short tandem repeats of genomic DNA which are characteristic to certain cancers and some other health conditions.

### BACKGROUND

The materials described in this section are not admitted to be prior art by inclusion in this section.

Cancer is a major health risk in the United States and internationally. The majority of current cancer therapies are focused on the patient's illness rather than targeted to individual patients. However, inter-individual differences in drug disposition and/or pharmokinetics have led to heterogeneity in patient responses to traditional cancer chemotherapy. As a result, over the last decade, cancer therapy has changed from a general chemotherapy based therapy in combination with surgery and radiation to a more personalized treatment that takes into account the genetic variability of tumors across patients. Currently, treatment plans often require identification of molecular alterations that allow a more targeted therapy. In many cases, such information is obtained by analysis of various molecules, such as, for example, nucleic acids and proteins from cancer tissue biopsies. However, tissue biopsies are often limited to initial diagnosis or surgery, and later biopsies tend to incur significant risk and discomfort to the patient. Moreover, tumor tissue biopsies tend to be problematic in terms of sampling bias and limited ability to monitor nucleic acid molecules as tumor markers in patients during the course of therapy.

In addition, a major challenge in cancer treatment is to identify patients early in the course of their disease. This is difficult under current diagnostic methodologies because early cancerous or precancerous cell populations may be asymptomatic and may be located in regions which are difficult to access by biopsy. Thus a robust, minimally invasive assay that may be used to identify all stages of the disease, including early stages which may be asymptomatic, would be of substantial benefit for the treatment of cancer.

### SUMMARY

This section provides a general summary of the disclosure, and is not comprehensive of its full scope or all of its feature.

In the past decade, several scientific studies have identified microsatellite instability (MSI) testing as a useful strategy for identification of patients who may have cancer with DNA mismatch repair deficiency and therefore may benefit from a treatment regimen with one or more immune checkpoint inhibitors. In a typical existing procedure, the molecular testing of MSI is performed on DNA samples extracted from fresh, frozen, or paraffin-embedded tumor tissue using a PCR-based assay. However, traditional biopsy methods can be invasive or dangerous for patients, often qualifying as surgery, and can include tissue biopsies (e.g., tissue, marrow), and imaging scans (x-ray, Mill, ultrasound). For example, medical imaging procedures often expose the patient to dangerous elements (e.g., radioactive materials). Alternatively, tissue biopsies can involve extracting relevant samples of matter from the body. These tissues may come directly from a tumor, such as in breast or skin cancer, or may be samples collected from bone marrow. While a specialized needle is often used to complete extraction, endoscopic methods may be used to extract tissue from unreachable areas, such as the lungs or colon, albeit in a manner perceivably more painful to the patient. Furthermore, should the tumor be located in an unreachable location due to another disease, as noted with obstructive pulmonary disease (COPD) and chronic kidney disease (CKD), tissue biopsy methods cannot be used to diagnose and track the tumor. Repeated use of traditional biopsy may also cause long-term damage to patients in a weak state of health, especially when discussing cancer in children.

The materials and methods of the present disclosure generally are designed for use in the analysis of microsatellite instability in CFNAs derived from various sources, including bodily fluids such as, e.g., blood samples. In in the past decade, analysis of cell-free nucleic acids (CFNAs) has been deployed for a wide variety of applications, including prenatal testing, paternity testing, forensics work, and in the detection, screening, prognosis and monitoring of the efficacy of anticancer therapies. For example, changes in the levels of CFNAs have been associated with tumor burden and malignant progression, and therefore CFNAs have been used as biomarkers in cancer patients.

The methods disclosed herein represent a significant improvement over existing technology, bringing increased power of discrimination, precision, and throughput to the analysis of MSI and to the diagnosis of illness, such as cancer, related to such MSI. As described in greater detail below, embodiments of the disclosure provide robust, minimally invasive methods that can be used to diagnose and/or treat (1) cancer at early stages of development which may be asymptomatic, (2) cancer that poses few symptoms, (3) cancer that is difficult to distinguish from benign conditions, and/or (4) cancer that can be developing in an area of the body that may not be accessible to traditional biopsy assays.

The present disclosure relates generally to methods for detecting and/or treating cancer in a patient by assessing cell-free nucleic acids (CFNA) derived from various bodily fluid samples such as, e.g., blood samples from the patient. In particular, in some embodiments, the disclosed methods involve identifying molecular alterations associated with microsatellite instability (MSI) present in the cell-free nucleic acids, such as, for example sequence alteration and/or a fragment-size alteration occurred within microsatellite short tandem repeats of genomic DNA which are characteristic to certain cancers and some other health conditions. In some embodiments, the methods of the disclosure are particularly suitable for bodily fluid samples derived from patients having or suspected of having a DNA mismatch repair (MMR) deficient cancer.

In one aspect, disclosed herein is a method for selecting a patient having cancer who is predicted to have an increased responsiveness for a treatment regimen including at least one checkpoint inhibitor, the method includes (i) obtaining cell-free nucleic acids derived from a blood sample taken from a patient having or suspected of having a DNA mismatch repair (MMR) deficient cancer: (ii) identifying one or more molecular alterations associated with microsatellite instability (MSI) present in the cell-free nucleic acids; and (iii) selecting the patient as predicted to have an increased responsiveness to the treatment regimen if one or more of the molecular alterations is detected in the cell-free nucleic acids.

In another aspect, some embodiments disclosed herein relate to a method for predicting the outcome of a treatment regimen for a patient having cancer, including (i) obtaining cell-free nucleic acids derived from a blood sample taken from a patient having or suspected of having a DNA mismatch repair (MMR) deficient cancer; and (ii) identifying one or more molecular alterations associated with microsatellite instability (MSI) present in the cell-free nucleic acids; wherein the presence of the one or more molecular alterations in the cell-free nucleic acids is indicative of an increased responsiveness in the patient to a treatment regimen including at least one checkpoint inhibitor.

In another aspect, some embodiments disclosed herein relate to a method for selecting a treatment regimen for a patient having a cancer, including (i) obtaining cell-free nucleic acids derived from a blood sample taken from a patient having or suspected of having a DNA mismatch repair (MMR) deficient cancer; (ii) identifying one or more molecular alterations associated with microsatellite instability (MSI) present in the cell-free nucleic acids; and (iii) selecting an appropriate treatment regimen for the treatment of the cancer in the patient based at least in part on whether one or more of the molecular alterations is present in the cell-free nucleic acids, wherein the treatment regimen includes at least one checkpoint inhibitor.

In yet another aspect, some embodiments disclosed herein relate to a method for treating a cancer in a patient, including (i) acquiring knowledge of the presence of one or more molecular alterations associated with microsatellite instability (MSI) present in cell-free nucleic acids derived from a blood sample taken from a patient having or being suspected of having a DNA mismatch repair (MMR) deficient cancer; (ii) selecting a therapeutic agent including at least one checkpoint inhibitor appropriate for the treatment of the cancer in the patient based at least in part on whether one or more of the molecular alterations is present in the cell-free nucleic acids; and administering a therapeutically effective amount of the selected therapeutic agent to the patient.

In yet another aspect, some embodiments disclosed herein relate to a method for treating a cancer patient, including (1) determining whether a patient has an increased responsiveness for a treatment regime including at least one checkpoint inhibitor by: (i) obtaining cell-free nucleic acids derived from a blood sample taken from a patient having or suspected of having a DNA mismatch repair (MMR) deficient cancer; (ii) identifying one or more molecular alterations associated with microsatellite instability (MSI) present in the cell-free nucleic acids; and (2) administering a therapeutic agent including at least one checkpoint inhibitor appropriate for the treatment of the cancer in the patient if one or more of the molecular alterations is detected in the cell-free nucleic acids.

In another aspect, some embodiments disclosed herein relate to a method for treating a cancer patient, including (1) determining whether a therapeutic agent including at least one checkpoint inhibitor is appropriate for cancer treatment by: (i) obtaining cell-free nucleic acids derived from a blood sample taken from a patient having or suspected of having a DNA mismatch repair (MMR) deficient cancer; (ii) identifying one or more molecular alterations associated with microsatellite instability (MSI) present in the cell-free nucleic acids; and administering a therapeutic agent including at least one checkpoint inhibitor appropriate for the treatment of the cancer in the patient if one or more of the molecular alterations is detected in the cell-free nucleic acids.

Implementations of embodiments of the methods as disclosed herein can include one or more of the following features. In some embodiments, the cell-free nucleic acids include one or more of circulating-free tumor DNAs, circulating-free tumor RNAs, and combinations of any thereof. In some embodiments, the identification of one or more molecular alterations includes an analytical assay selected from the group consisting of electrophoresis, chromatography, centrifugation, nucleic acid sequencing, genomic sequencing, next-generation sequencing (NGS), nucleic acid amplification-based assays, nucleic acid hybridization assays, polymerase chain reaction (PCR) assay, real-time PCR assay, quantitative reverse transcription PCR (qRT-PCR) assay. In some embodiments, the identification of one or more molecular alterations includes an enrichment process, based on size discrimination, to produce an enriched fraction of cell-free nucleic acids of about 1,200 base pairs or less. In some embodiments, the enrichment includes electrophoresis. In some embodiments, the electrophoresis includes capillary electrophoresis. In some embodiments, the enrichment process includes centrifugation. In some embodiments, the centrifugation includes gradient centrifugation. In some embodiments, the enrichment includes chromatography. In some embodiments, the chromatography includes high performance liquid chromatography (HPLC). In some embodiments, the enrichment produces an enriched fraction of cell-free nucleic acid of about 500 base pairs or less.

In some embodiments of the methods disclosed herein, the one or more molecular alterations associated with MSI is selected from the group consisting of an aberrant production of the cell-free nucleic acids, a sequence alteration of short tandem DNA repeats, and a fragment-size alteration within short tandem DNA repeats. In some embodiments, the one or more molecular alterations associated with MSI includes a fragment-size alteration within microsatellite short tandem repeats. In some embodiments, the one or more molecular alterations associated with MSI includes aberrant level of cell-free nucleic acids derived from one or more microsatellite loci in the patient. In some embodiment, the identification of one or more molecular alterations includes generating a profile of microsatellite markers for the patient.

In some embodiments, the methods disclosed herein further include detecting one or more genetic alterations in a target gene known to be associated with microsatellite instability (MSI) in the blood sample. In some embodiments, the target gene is a DNA mismatch repair (MMR) gene. In some embodiments, the DNA mismatch repair (MMR) gene is selected from the group consisting of Msh2, Msh3, Msh4, Msh5, Msh6, Mlh1, Mlh3, Pms1, Pms2, Exo1, Pol δ, PNCA, RPA, HMGB1, RFC, and DNA ligase I. In some embodiments, the target gene is a gene associated with checkpoint inhibition. In some embodiments, the target gene associated with checkpoint inhibition is selected from the group consisting of PD-1, CTLA-4, A2AR, B7-H3, B7-H4 s, BTLA, IDO, KIR, LAG3, TIM-3, and VISTA.

In some embodiments of the methods disclosed herein, the one or more genetic alterations is selected from the group consisting of a genetic mutation, a gene amplification, a gene rearrangement, a single-nucleotide variation (SNV), a deletion, an insertion, an In/Del mutation, a single nucleotide point mutation (SNP), an epigenetic alteration, a splicing variant, an RNA/protein overexpression, an aberrant RNA/protein expression, genomic instability, genomic rearrangement, and any combination thereof. In some embodiments, the detection of one or more genetic alterations includes an analytical assay selected from the group consisting of electrophoresis, nucleic acid sequencing, nucleic acid amplification-based assays, nucleic acid hybridization assays, polymerase chain reaction (PCR) assay, real-time PCR assay, quantitative reverse transcription PCR (qRT-PCR) assay, genomic sequencing, next-generation sequencing (NGS). In some embodiments, the one or more genetic alterations results in a sequence alteration and/or a fragment-size alteration within microsatellite short tandem repeats.

In some embodiments of the disclosure, the methods disclosed herein include (i) identifying a fragment-size alteration within microsatellite short tandem repeats; and (2) identifying one or more genetic alterations in an MMR gene.

In some embodiments of the methods disclosed herein, the at least one checkpoint inhibitor is selected from the group consisting of PD-1 inhibitors, CTLA-4 inhibitors, A2AR inhibitors, B7-H3 inhibitors, B7-H4 inhibitors, BTLA inhibitors, IDO inhibitors, KIR inhibitors, LAG3 inhibitors, TIM-3 inhibitors, VISTA inhibitors, and combinations of any thereof. In some embodiments, the at least one checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C, and combinations thereof.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### DETAILED DESCRIPTION OF THE DISCLOSURE

The present disclosure relates generally to the field of molecular biology and medicine, including methods for detecting and/or treating cancer by assessing circulating free nucleic acids derived from suitable bodily fluids such as, for example, blood samples. In some embodiments, the present disclosure provides for, inter alia, methods for detecting and/or treating cancer in a cancer patient by analyzing cell-free nucleic acids (CFNAs) present in any one of various bodily fluids such as, e.g., blood samples taken from the patient. In some embodiments of the disclosure, the bodily fluid is whole blood and/or a cell-depleted fraction thereof (e.g., plasma or serum). In particular, the disclosed methods according to some embodiments of the disclosure include identifying molecular alterations associated with microsatellite instability (MSI) present in the CFNAs, such as, for example, the presence of one or more sequence alterations and/or a fragment-size alterations within microsatellite short tandem repeats of the genomic DNA, which are characteristic to certain cancers and some other health conditions. In some embodiments of the disclosed methods, the methods of the disclosure are particularly suitable for patients having or suspected of having a DNA mismatch repair (MMR) deficient cancer.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal subject, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In some embodiments, the cancer is a DNA mismatch repair (MMR) deficient cancer.

The terms “subject,” “patient,” “individual in need of treatment” and like terms are used interchangeably and refer to, except where indicated, an mammal subject that is the object of treatment, observation, or experiment. In some embodiments, the mammal is a human. However, in some embodiments, the mammal is not a human. The term does not necessarily indicate that the subject has been diagnosed with a particular disease or disorder, but typically refers to a subject under medical supervision. “Subject suspected of having” means a subject exhibiting one or more clinical indicators of a disease or condition. In certain embodiments, the disease or condition is a cancer.

As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented. In some embodiments of the disclosure, the terms “treatment,” “therapy,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, increase in survival time or rate, etc., or a combination thereof. The effect of treatment can be compared to an individual or pool of individuals not receiving the treatment, or to the same patient prior to treatment or at a different time during treatment. In some embodiments, the severity of disease or disorder in an individual can be reduced by at least 10%, as compared, e.g., to the individual before administration or to a control individual not undergoing treatment. In some embodiments, the severity of disease or disorder in an individual is reduced by at least 25%, 50%, 75%, 80%, or 90%, or in some embodiments, no longer detectable using standard diagnostic techniques.

As used herein, the term “effective amount” or “therapeutically effective amount” refers to an amount sufficient to effect beneficial or desirable biological and/or clinical results. In some embodiments, the term refers to that amount of the therapeutic agent sufficient to ameliorate a given disorder or symptoms. For example, for the given parameter, a therapeutically effective amount can show an increase or decrease of at least 5%, 10%, 15%, 20%, 25%, 40%, 50%, 60%, 75%, 80%, 90%, or at least 100% compared to a control. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

In some embodiments of the methods or processes described herein, the steps can be carried out in any order, except when a temporal or operational sequence is explicitly recited. Furthermore, in some embodiments, the specified steps can be carried out concurrently unless explicit claim language recites that they be carried out separately. For example, in some embodiments a claimed step of doing X and a claimed step of doing Y can be conducted simultaneously within a single operation, and the resulting process will fall within the literal scope of the claimed process.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

### Microsatellite Instability (MSI)

Microsatellite structure consists of short repeated nucleotide sequences, most often seen as GT/CA repeats. These sequences can be made of repeating units of one to six base pairs in length. These repeats are distributed throughout the genome in all genomic components (e.g., coding sequences, untranslated regions, introns and intergenic spaces) and often vary in length from one individual to another, due to differences in the number of tandem repeats at each locus. Typically, a microsatellite locus is defined as a region of genomic DNA with simple tandem repeats that are repetitive units of one to six base pairs in length. It has been well documented that hundreds of thousands of such microsatellite loci are dispersed throughout the human genome. Microsatellite loci are generally classified based on the length of the smallest repetitive unit. For example, loci with repetitive units of 1 to 6 base pairs in length are termed “mononucleotide”, “dinucleotide”, “trinucleotide”, “tetranucleotide”, “pentanucleotide”, and “hexanucleotide” repeat loci, respectively. Among the most common microsatellites in humans is a dinucleotide repeat of the nucleotides C and A, which occurs tens of thousands of times across the genome. In some cases, microsatellites are also known as simple sequence repeats (SSRs). Although the length of these microsatellites is highly variable from person to person and contributes to the individual DNA “fingerprint”, it is generally believed that each individual has microsatellites of a set length.

Each microsatellite locus of normal genomic DNA for most diploid species, such as genomic DNA from mammalian species, consists of two alleles at each locus. The two alleles can be the same or different from one another in length and can vary from one individual to the next. Microsatellite alleles are normally maintained at constant length in a given individual and its descendants; however, instability in the length of microsatellites has been observed in some tumor types. This form of genomic instability in tumors, termed microsatellite instability (hereinafter, “MSI”), is a molecular hallmark of several cancer syndromes. Generally, MSI is characterized as a change in sequence or in length of a microsatellite allele due to insertion or deletion of repeat units during DNA replication and failure of the DNA mismatch repair system to correct these errors. For example, MSI has been found in over 90% of Hereditary Nonopolyposis Colorectal Cancer (HNPCC) and in 10-20% of sporadic colorectal tumors. However, MSI is not limited to colorectal tumors. In fact, MSI has also been detected in pancreatic cancer, gastric cancer, leukemia, colorectal cancer, lung cancer, prostate cancer, brain tumors, central nervous system tumors, bladder tumors, melanomas, liver cancer, bone cancer, testicular carcinoma, ovarian carcinoma, head and neck tumors, and cervical cancer.

Recently, numerous microsatellite markers have been developed and used to detect MSI. Several methodologies and systems known in the art that can be suitably deployed for the assessment of MSI status in genomic loci of interest. For example, in some embodiments of the disclosure, insertion or deletion of one or more repetitive units during DNA replication persists without mismatch repair and can be detected as length polymorphisms by comparison of allele sizes found in microsatellite loci amplified from normal and tumor DNA samples, using any one of several methodologies and systems known in the art. These methodologies and systems typically include PCR based techniques. For example, various commercially available test kits and services for MSI analysis are offered by Promega Corporation (Cat #MD1641), Thermo Fisher Scientific, and NeoGenomics. Alternatively, MSI may also be inferred from a specific omics analysis as is described in U.S. Patent Publication No. 2017/0032082A1.

Typically, MSI analysis involves comparing allelic profiles of microsatellite markers generated by amplification of DNA from matching normal and test samples, which may be derived from patients having or suspected of having a DNA mismatch repair (MMR) deficient cancer. Alleles that are present in the test sample but not in corresponding normal samples indicate MSI. Generally, mononucleotide repeat markers included in MSI analysis are selected for high sensitivity and specificity to alterations in samples containing mismatch repair defects, and most preferably such mononucleotide repeat markers are quasi-monomorphic (e.g., almost all individuals (e.g., at least 90%, more typically at least 95% of individuals) are homozygous for the same common allele for a given marker). As will be readily appreciated by one of ordinary skill in the art upon review of this disclosure, use of quasi-monomorphic or monomorphic markers simplifies data interpretation, and there are numerous suitable genetic markers, e.g., loci, known in the art to identify MSI. For example, suitable genetic markers and loci are described in U.S. Pat. Nos. 6,150,100 and 7,662,595; U.S. Patent Publication Nos. 2003/0113723A1 and 2015/0337388A1; and PCT Patent Publication No. WO 2017/112738.

### DNA-Mismatch Repair (MMR)

It has been documented that microsatellite instability is the condition of genetic hypermutability (predisposition to mutation) that typically results from impaired DNA mismatch repair (MMR) pathway. The MMR pathway, which is found to be highly conserved from bacteria to humans, targets base substitution mismatches and insertion-deletion mismatches (IDLs) that arise as a result of replication errors that escape the proofreading function of DNA polymerases. In doing so, MMR machinery is believed to contribute an additional 50-1000-fold to the overall fidelity of DNA replication. In its role in post-replication repair, MMR pathway safeguards the genome by correcting base mispairs arising as a result of replication errors. Loss of MMR functions results in greatly increased rates of spontaneous mutation in organisms ranging from bacteria to humans.

Without being bound to any particular theory, it is believe that repetitive DNA is particularly sensitive to errors in replication and therefore dysfunctional mismatch repair systems result in widespread alterations in microsatellite regions. Several studies of yeast cells without functional mismatch repair systems showed a 2800-, 284-, 52-, and 19-fold increase in mutation rates in mono-, di-, tri-, tetra-, and pentanucleotide repeats, respectively. Mutations in mismatch repair genes are not thought to play a direct role in tumorigenesis, but rather act by allowing DNA replication errors to persist. MMR-deficient cells have high mutation rates and if these mutations occur in genes involved in tumorigenesis the cell may become cancerous. As discussed above, a hallmark of many MMR-deficient cells is instability at microsatellite regions consisting of mono- and di-nucleotide repeats. Strand slippage during replication through microsatellite regions gives rise to IDLs that are normally repaired by MMR; hence, microsatellite instability (MSI) is widely used as a diagnostic marker for loss of MMR activity in tumor cells.

It is also generally believed that MMR corrects errors that spontaneously occur during DNA replication, such as single base mismatches or short insertions and deletions. The proteins involved in MMR correct polymerase errors by forming a complex that binds to the mismatched section of DNA, excising the error, and inserting the correct sequence in its place. Cells with abnormally functioning MMR are unable to correct errors that occur during DNA replication and consequently accumulate errors. This causes the creation of novel microsatellite fragments. In these case, polymerase chain reaction-based assays and sequencing assays can be used to reveal these novel microsatellites and provide evidence for the presence of MSI. About 150 human DNA repair genes have been identified (Wood R D et al., Sciences, Vol. 577, Issues 1-2, 4 Sep. 2005, pp. 275-283; and Feb. 16 2001; 291(5507):1284-9), including genes encoding DNA repair enzymes, some genes associated with cellular responses to DNA damage, and other genes associated with genetic instability or sensitivity to DNA damaging agents.

Table 1 summarizes examples of animal models for MMR deficiency in which each MMR gene has been knocked out confirm that loss of MMR confers a mutator phenotype (MSI+), increased incidence of cancer and decreased lifespan. In Table 1, gastrointestinal (GI) tumors include adenoma, adenocarcinoma, and flat adenoma in gastrointestinal organs; skin tumors include Squamous cell carcinoma, keratoacanthoma, sebaceous (adenoma, epithelioma, carcinoma), papilloma, haemangioma, and pylomatricoma; other tumors include tumors of the uterus, brain, lung, liver, and mammary gland; sarcoma include Leiomyosarcoma, myxoid sarcoma, and fibroid sarcoma. Typically, MSI is analyzed in tumor samples, normal tissue or culture cells. Tumor spectrum includes lymphoma: B and T cell lymphoma; NHL is non-Hodgkin's lymphoma.

TABLE 1
Mouse models with mutations in MMR genes

| | Tumour | | MSI incidence^{a} | | Fertility |
|---|---|---|---|---|---|
| Genotype | Spectrum^{b} | Incidence | Mononucleotide | Dinucleotide | Male/female |
| MutS homologues | | | | | |
| MSH2^{-/-} | Lymphoma, | High | High | High | +/+ |
| | GI, skin and | | | | |
| | other tumours | | | | |
| MSH2 C674A/C674A | Lymphoma, | High | High | High | +/+ |
| | GI, skin | | | | |
| MSH3^{-/-} | GI tumours | Low | Moderate | High | +/+ |
| MSH6^{-/-} | Lymphoma, | High | None | Low | +/+ |
| | GI, skin and | | | | |
| MSH6 TI217D/TI217D | Lymphoma, | High | High | High | +/+ |
| | GI, skin | | | | |
| MSH3^{-/-} MSH6^{-/-} | Lymphoma, | High | High | High | +/+ |
| | GI, skin and | | | | |
| | other tumours | | | | |
| MSH4^{-/-} | None | None | N/A | N/A | −/− |
| MSH5^{-/-} | None | None | N/A | N/A | −/− |
| MutL homologues | | | | | |
| MLH1^{-/-} | Lymphoma, | High | High | High | −/− |
| | GI, skin and | | | | |
| | other tumours | | | | |
| PMS1^{-/-} | None | None | Low | Low | +/+ |
| PMS2^{-/-} | Lymphoma and | High | High | High | −/+ |
| | sarcoma | | | | |
| MLH3^{-/-} | N/A | N/A | Low | Low | −/− |
| Exonuclease | | | | | |
| EXO1^{-/-} | Lymphoma | Moderate | High | Low | −/− |

For example, gene disruption of EXO1 in mice results in MMR defects, increased cancer susceptibility, and male and female sterility. The repair defect in EXO1−/− cells also caused elevated microsatellite instability at a mononucleotide repeat marker and a significant increase in mutation rate. EXO1−/− mice displayed reduced survival, increased susceptibility to lymphomas and meiotic defects. In another example, a hallmark of Hereditary Nonpolyposis Colorectal Cancer (HNPCC) or Lynch Syndrome is microsatellite instability. This is because microsatellites are particularly susceptible to DNA replication errors when the MMR system is absent. In particular, frameshift mutations of the microsatellite repeats in the TGF/RII coding region were found in ˜90% of HNPCC tumors. Five markers (BAT26, BAT25, D5S346, D2S123, and D17S250) are recommended by the National Cancer Institute to assess microsatellite instability. Without being bound any particular theory, the observation of alterations of key growth regulatory genes in MMR-deficient cells such as NF1, APC, p53, K-Ras suggest that even in the presence of MSI, tumor progression is mainly driven by a process of natural selection. MSI+ colorectal carcinoma has been associated with a more favorable clinical outcome that has confounded clinical studies assessing drug efficacy among colorectal cancer patients. Early attempts at identifying the genetic basis for HNPCC revealed frequent insertions and deletions at di- and trinucleotide repeats or microsatellite regions at a putative HNPCC locus on human chromosome 2 as well as throughout the genome. Compared with MMR-proficient tumors, MMR-deficient tumors (MSI+) tend to be proximal to splenic flexure, poorly differentiated, mucinous, characterized by marked lymphocyte infiltration, and frequently large in size. MSI+ was also strongly associated with a decreased likelihood of lymph node and distant organ metastases.

In another example, several mutations in MMR genes have been reported to cause hereditary nonpolyposis colorectal cancer, and loss of MMR function is associated with a significant fraction of sporadic cancer. In this case, MSI positive tumors have been found to carry somatic frameshift mutations in mononucleotide repeats in the coding region of several genes involved in growth control, apoptosis, and DNA repair (e.g., TGFBRII, BAX, IGFIIR, TCF4, MSH3, MSH6). Among the most commonly altered locus is TGFBRII, in which over 90% of MSI-H colon tumors have been found to contain a mutation in the 10 base polyadenine repeat present in the gene.

Taken together, inactivation of MMR machinery confers a strong mutator phenotype in which the rate of spontaneous mutation is greatly elevated. Therefore, the presence of MSI is generally considered as evidence that MMR machinery is not functioning normally.

### Methods of the Disclosure

The present disclosure relates generally to methods for detecting and/or treating cancer in a patient by analyzing cell-free nucleic acids (CFNA) derived from various bodily fluid samples such as, e.g., blood samples from the patient. In particular, in some embodiments, the disclosed methods involve identifying molecular alterations associated with microsatellite instability (MSI) present in the cell-free nucleic acids, such as, for example alterations in the nucleic acid sequence of microsatellite short tandem repeats and/or fragment-size alterations within microsatellite short tandem repeats. These types of molecular alterations are characteristic to certain cancers and some other health conditions. In some embodiments of the disclosure, the bodily fluid samples are derived from patients having cancer or are suspected of having a DNA mismatch repair (MMR)-deficient cancer.

The bodily fluid sample relevant for the present disclosure can generally be any bodily fluid samples known to contain cell-free nucleic acids, and can be, for example, amniotic fluid, blood, plasma, serum, and semen. Other non-limiting examples of bodily fluid samples that are suitable for the methods disclosed herein include, but are not limited to, lymphatic fluid, follicular fluid, cerebral spinal fluid, ocular fluid, urine, saliva, mucous, and sweat. In some particular embodiments, the bodily fluid sample includes blood or blood components. In some embodiments, the bodily fluid sample includes whole blood. In some embodiments, the sample includes a cell-free fraction of a bodily fluid sample, such as blood plasma. In some embodiments, the bodily fluid sample includes one or more blood components such as, for example, plasma or serum.

In one aspect, some embodiments of the present disclosure relate to methods for selecting a patient having cancer who is predicted to have an increased responsiveness for a treatment regimen comprising administration of at least one checkpoint inhibitor, the method includes (i) obtaining cell-free nucleic acids (CFNAs) derived from a blood sample taken from a patient having or suspected of having a DNA mismatch repair (MMR) deficient cancer: (ii) identifying one or more molecular alterations associated with microsatellite instability (MSI) present in the CFNAs; and (iii) selecting the patient as predicted to have an increased responsiveness to the therapeutic treatment if one or more of the molecular alterations is detected in the CFNAs.

In another aspect, some embodiments of the disclosure relate to methods for predicting the outcome of a treatment regimen for a patient having cancer, including (i) obtaining cell-free nucleic acids derived from a blood sample taken from a patient having or suspected of having a DNA mismatch repair (MMR) deficient cancer; and (ii) identifying one or more molecular alterations associated with microsatellite instability (MSI) present in the cell-free nucleic acids; wherein the presence of the one or more molecular alterations in the cell-free nucleic acids is indicative of an increased responsiveness in the patient to a treatment regimen including at least one checkpoint inhibitor.

In yet another aspect, some embodiments disclosed herein relate to methods for selecting a treatment regimen for a patient having a cancer, including (i) obtaining cell-free nucleic acids derived from a blood sample taken from a patient having or suspected of having a DNA mismatch repair (MMR) deficient cancer; (ii) identifying one or more molecular alterations associated with microsatellite instability (MSI) present in the cell-free nucleic acids; and (iii) selecting an appropriate treatment regimen for the treatment of the cancer in the patient based at least in part on whether one or more of the molecular alterations is present in the cell-free nucleic acids, wherein the treatment regimen includes at least one checkpoint inhibitors.

The term “cell-free nucleic acid” or “circulating free nucleic acids”, as used interchangeably herein, refers to extracellular nucleic acids present in a bodily fluid sample. Cell-free nucleic acids (CFNAs) are also often referred to as extracellular nucleic acids that are not contained in cells. CFNAs can be found in bodily fluids such as blood, plasma, serum, semen, amniotic fluid, lymphatic fluid, follicular fluid, cerebral spinal fluid, ocular fluid, urine, saliva, mucous, and sweat. In some embodiments, the bodily fluid sample comprising the CFNAs is a cell-free or cell-depleted sample. A respective cell-free or cell-depleted sample can be obtained, e.g., from a cell-containing sample by using appropriate technologies to remove cells. For example, a typical cell-depleted sample is blood plasma or blood serum which can be obtained from whole blood. If the sample comprises large amounts of cells as is the case with whole blood, the cells are separated from the remaining sample in order to obtain a cell-free, respectively cell-reduced fraction of the sample which comprises the CFNAs. Thus, according to one embodiment, cells are removed from the cell-containing sample to provide the cell-free or cell-depleted sample which comprises the CFNAs and from which the CFNAs are isolated. Depending on the sample type, cells, including residual cells, can be separated and removed by, e.g., centrifugation, preferably high speed centrifugation, or by using means other than centrifugation, such as, e.g., filtration, sedimentation or binding to surfaces on (optionally magnetic) particles if a centrifugation step is to be avoided.

In some embodiments of methods disclosed herein, the term “cell-free nucleic acid” encompasses cell-free DNA (cfDNA) and/or cell-free RNA (cfRNA). Accordingly, in some embodiments of the methods disclosed herein, the CFNAs include one or more of cfDNA, cfRNA, and combinations of any thereof. Without being bound to any particular theory, CFNAs in the bodily fluid sample may result from the shedding of nucleic acids from cells undergoing apoptosis or necrosis. Previous studies have demonstrated that CFNAs, for example cfDNA, exists at steady-state levels and can increase with cellular injury or necrosis. In some embodiments of the disclosure, CFNA is shed from abnormal cells or unhealthy cells, such as tumor cells. cfDNA shed from tumor cells, in some embodiments, can be distinguished from cfDNA shed from normal or healthy cells using genomic information, such as by identifying genetic variations including mutations and/or gene fusions distinguishing between normal and abnormal cells. In some embodiments, CFNA is shed into maternal circulation from cells associated with a fetus. Accordingly, in some embodiments of the disclosure, the term CFNAs in particular refers to mammalian CFNAs, preferably disease-associated or disease-derived CFNAs such as tumor-associated or tumor-derived CFNAs, or CFNAs released due to inflammations or injuries, in particular traumata, CFNAs related to and/or released due to other diseases, or CFNAs derived from a fetus.

In particular embodiments where the CFNAs encompass cfRNA, the cfRNA can include full length RNA as well as fragments of full length RNA (which can have a length of 50-150 bases, 15-500 bases, or 500-1,000 bases, or more). Thus, cfRNA can represent a portion of an RNA, which can be between about 100-80% of the full length RNA (typically mRNA), or between about 90-70%, or between about 80-60%, or between about 70-50%, or between about 60-40%, or between about 50-30%, or between 40-20% of the full length RNA. In some embodiments, the CFNA includes nucleic acids derived from a tumor cell (as opposed to nucleic acids derived from a non-tumor cell) and that the CFNA can therefore be from a tumor cell of a solid tumor, a blood borne cancer, circulating tumor cells, and/or exosomes. Typically, the CFNA in accordance with some embodiments of the present disclosure is not enclosed by a membrane (and as such be from a circulating tumor cell or exosome). In some embodiments, the CFNAs can be transcripts uniquely expressed in a tumor (e.g., as a function of drug resistance or in response to a treatment regimen, as a splice variant, etc.) or as a mutated form of a gene (e.g., as a fusion transcript, as a transcript of a gene having a single or multi-base mutation, etc.). Therefore, the CFNAs in some embodiments of the present disclosure include transcripts that are unique to a tumor cell relative to a corresponding non-tumor cell, or significantly over-expressed (e.g., at least 3-fold, or at least 5-fold, or at least 10-fold) in a tumor cell relative to a corresponding non-tumor cell, or have a mutation (e.g., missense or nonsense mutation leading to a neoepitope) relative to a corresponding non-tumor cell.

The bodily fluid of the patient can be obtained at any desired time point(s) depending on the purpose of the CFNA analysis. For example, the bodily fluid of the patient can be obtained before and/or after the patient is confirmed to have a tumor and/or periodically thereafter (e.g., every week, every month, etc.) in order to associate the cell-free DNA/RNA data with the prognosis of the cancer and MSI status. Thus, in some embodiments, the bodily fluid of the patient can be obtained before the patient is confirmed to have a tumor. In some embodiments, the bodily fluid can be obtained from a patient suspected of having a tumor. In some embodiments, the bodily fluid of the patient can be obtained after the patient is confirmed to have a tumor.

Additionally or alternatively, the bodily fluid of a healthy individual can be obtained to compare the sequence/modification of cell-free DNA, and/or quantity/subtype expression of cell-free RNA. As used herein, a healthy individual refers an individual without a tumor. In some preferred embodiments, the healthy individual can be chosen among group of people shares characteristics with the patient (e.g., age, gender, ethnicity, diet, living environment, family history, etc.).

In some embodiments of the methods disclosed herein, the bodily fluid of the patient can be obtained from a patient before and after the cancer treatment (e.g., before/after chemotherapy, radiotherapy, drug treatment, cancer immunotherapy, etc.). While it may vary depending on the type of diagnostic assays, treatments, and/or the type of cancer, the bodily fluid of the patient can be obtained at least 1 month, 3 weeks, 2 weeks, 1 week, at least 5 days, at least 4 days, 72 hours, 48 hours, 24 hours, at least 12 hours, 6 hours, 3 hours, 1 hour before and/or after the diagnostic assay or the cancer treatment. For more accurate comparison, the bodily fluid from the patient can be obtained less than 1 hour before, less than 3 hours before, less than 6 hours before, less than 8 hours before, less than 12 hours before, less than 24 hours before, less than 48 hours before, less than a week before and/or after the diagnostic assay or the cancer treatment. In addition, a plurality of samples of the bodily fluid of the patient can be obtained during a period before and/or after the diagnostic assay or the cancer treatment (e.g., once a day after 24 hours for 7 days, etc.).

Isolation and Amplification of Cell-Free Nucleic Acids (CFNA)

The isolation and/or amplification of cell-free nucleic acids (CFNA) according to methods disclosed herein can be performed by using any one of suitable methodologies and systems known in the art. In some embodiments, CFNA can be isolated from a bodily fluid (e.g., whole blood) and/or a cell-depleted fraction thereof (e.g., plasma) that is processed under suitable conditions, including a condition that stabilizes CFNA. In some embodiments, both cell-free DNA and cell-free RNA are isolated simultaneously from the same sample of the patient's bodily fluid and/or a cell-depleted fraction thereof. Yet, in some embodiments, the bodily fluid sample and/or a cell-depleted fraction thereof can be divided into two or more smaller samples from which cell-free DNA or cell-free RNA can be isolated separately. It will be readily appreciated by the skilled artisan that numerous other collection modalities are also deemed appropriate, and that the cell-free RNA can be at least partially purified or adsorbed to a solid phase to so increase stability prior to further processing. Similarly, cell-free DNA can be isolated using commercially available reagents and methods, and kits including Cell-Free DNA BCT® (Streck Inc., USA); MagNA Pure Compact (MPC) Nucleic Acid Isolation Kit I (Roche Diagnostics, Germany); and Maxwell® RSC (MR) cfDNA Plasma Kit (Promega Corporation, USA); and the QIAamp® Circulating Nucleid Acid (QCNA) Kit (QIAgen, USA). Suitable methods and kits are also commercially available such as the QIAamp MinElute Virus Spin or Vacuum Kit (QIAGEN), the Chemagic Circulating NA Kit (Chemagen), the NucleoSpin Plasma XS Kit (Macherey-Nagel), the Plasma/Serum Circulating DNA Purification Kit (Norgen Biotek), the Plasma/Serum Circulating RNA Purification Kit (Norgen Biotek), the High Pure Viral Nucleic Acid Large Volume Kit (Roche) and other commercially available kits suitable for purifying cell-free nucleic acids.

In one exemplary method of cell-free DNA isolation, specimens can be collected as 10 ml of whole blood drawn into a test tube. Cell-free DNA can be isolated from other from mono-nucleosomal and dinucleosomal complexes using magnetic beads that can separate out cell-free DNA at a size between 100-300 bps. In another exemplary method of RNA isolation, specimens can be collected as 10 ml of whole blood drawn into cell-free RNA BCT® tubes or cell-free DNA BCT® tubes containing RNA stabilizers, respectively. In some instances, cell-free RNA is stable in whole blood in the cell-free RNA BCT tubes for seven days while cell-free RNA is stable in whole blood in the cell-free DNA BCT Tubes for fourteen days, allowing time for shipping of patient samples from world-wide locations without the degradation of cell-free RNA. Moreover, one of ordinary skill in the art will appreciate that cell-free RNA can be isolated using RNA stabilization agents that will not or substantially not lyse blood cells (e.g., equal or less than 1%, or equal or less than 0.1%, or equal or less than 0.01%, or equal or less than 0.001% of blood cells are lysed). In addition or alternatively, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation can be in form of collected blood that may or may not have been separated. In another embodiment, the RNA stabilization reagents will stabilize cell-free RNA in a collected tissue other than blood for at 2 days, more preferably at least 5 days, and most preferably at least 7 days.

As will be readily appreciated by one skilled in the art, fractionation of plasma and extraction of cell-free DNA/RNA can be achieved by several methodologies and strategies. For example, whole blood samples collected in 10 mL tubes are centrifuged to fractionate plasma at 1600 rcf for 20 minutes. The so obtained plasma are then separated and centrifuged at 16,000 rcf for 10 minutes to remove cell debris. Various alternative centrifugal protocols can be used and deemed suitable so long as the centrifugation do not lead to substantial cell lysis (e.g., lysis of no more than 1%, or no more than 0.1%, or no more than 0.01%, or no more than 0.001% of all cells). In some experiments, cell-free DNA/RNA can be extracted from plasma using Qiagen reagents. The extraction protocol can be designed to remove potential contaminating blood cells, other impurities, and maintain stability of the nucleic acids during the extraction. When needed, all nucleic acids are kept in bar-coded matrix storage tubes, with DNA stored at −4° C. and RNA stored at −80° C. or reverse-transcribed to cDNA that is then stored at −4° C. In addition, the isolated cell-free nucleic acids are optionally frozen prior to further processing.

Once separated from the non-nucleic acid components, the CFNAs can then optionally be analyzed using any one of known analytical assays to identify, detect, screen for, monitor or exclude a healthy condition or a cancer such as, for example, an MMR-deficient cancer. The analytical assays of the nucleic acids can generally be performed using any nucleic acid analytical assays and methodologies including, but not limited to amplification technologies, polymerase chain reaction (PCR), isothermal amplification, reverse transcription polymerase chain reaction (RT-PCR), quantitative real time polymerase chain reaction (Q-PCR), digital PCR, gel electrophoresis, capillary electrophoresis, mass spectrometry, fluorescence detection, ultraviolet spectrometry, hybridization assays, DNA or RNA sequencing, restriction analysis, reverse transcription, NASBA, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof.

By far the most common method to detect MSI is to measure the length of a polymerase chain reaction amplicon containing the entire microsatellite. This procedure typically requires CFNAs, a pair of primers of which one is often fluorescently end labeled, a sequencer, and suitable software. Alternatively, if the amplicon is sequenced, one can simply count the number of repeat units. MSI can also be indirectly diagnosed by detecting loss of staining by immunohistochemistry (IHC) of one of the mismatch repair genes, since this also points to an abnormality in mismatch repair. In practice, both immunohistochemical and genetic methods may produce a number of false-negatives, and for this reason combined assessments at the immunohistochemical and genetic level are typically performed in a routine diagnostic setting.

Accordingly, in some embodiments, the identification of one or more molecular alterations can include one or more analytical assays performed on the bodily fluid sample and/or on the cell-free nucleic acids derived therefrom. The analytical assay can generally be any analytical assay known to those having ordinary skill in the art, and can be for example an electrophoresis, chromatography, centrifugation, or a nucleic acid sequencing. Non-limiting examples of suitable analytical assays include, but are not limited to, genomic sequencing assays, next-generation sequencing (NGS), nucleic acid amplification-based assays, nucleic acid hybridization assays, polymerase chain reaction (PCR) assay, real-time PCR assay, and quantitative reverse transcription PCR (qRT-PCR) assay. In some embodiments, an electrophoretic assay is used to identify the one or more molecular alterations in the CFNAs. In some embodiments, the electrophoretic assay includes capillary electrophoresis.

MSI-Loci from Cell-Free Nucleic Acids (CFNA, Amplification, and Size Determination)

With respect to MSI loci and markers suitable for the methods disclosed herein, the MSI loci and markers can generally be selected from any known MSI loci and markers. However, particularly preferred MSI loci include mono- and dinucleotide repeat markers, and most preferably those associated mismatch repair defects. Thus, and viewed from a different perspective, contemplated repeat markers are quasi-monomorphic (e.g., almost all individuals (e.g., at least 90%, more typically at least 95% of individuals) are homozygous for the same common allele for a given marker). More information regarding MSI loci and markers suitable for the methods described herein can be found U.S. Pat. Nos. 6,150,100, 7,662,595, US 2003/0113723, US 2015/0337388, and WO 2017/112738, all of which are incorporated by reference herein. Non-limiting examples of repeats include NR-21, BAT-26, BAT-25, NR-27, NR-24, D2S123, D5S346, D17S250, BAT40, MONO-27, Penta C, Penta D, D18535, D1S2883, etc.

When nucleic acid amplification-based assays are used, amplification conditions can vary depending on the particular repeat sequence/MSI locus marker. In these instances, the particular PCR conditions for specific MSI loci and markers can be readily ascertainable. For example, PCR conditions and reagents for amplification of NR-21, BAT26, BAT-25, NR-24, and Mono-27 is described in the product manual for the commercially available MSI Analysis System from Promega Corp. In these instances, it will be readily appreciated by those skilled in the art that the amplification reagents can include fluorescence or otherwise labeled nucleotides, or can be performed without detectable markers. Therefore, the manner of detection will vary accordingly. In some embodiments, for size determination of the amplicons, it is contemplated that the amplified products will be subjected to a chromatographic step that provides sufficient resolution in the size range of the amplicons. For example, suitable fragment size determination can be performed using capillary electrophoresis (e.g., using ABI PRISM 310 or Applied Biosystems 3130 Genetic Analyzer), polyacrylamide gel electrophoresis, mass spectroscopy, chip-based microfluidic electrophoresis (Methods Mol Biol. 2013; 919:287-96), and denaturing high performance liquid chromatography. In some embodiments, size determination analysis is performed in parallel with a reference sample to detect a shift in allelic size distribution. In some embodiments, the reference sample is derived from an individual lacking the one or more molecular alternations. Fragment-size determination methodologies and suitable reagents and systems are known in the art.

In some embodiments of the methods disclosed herein, the identification of one or more molecular alterations can include an enrichment step, by enriching the fraction of CFNAs comprising one or more of the molecular alterations in the total CFNAs present in the bodily fluid sample. In some embodiments, the identification of one or more molecular alterations includes an enrichment step, based on size discrimination, to produce an enriched fraction of cell-free nucleic acids of about 1,200 base pairs or less in a high background of genomic nucleic acid. This leads to a relatively enriched fraction of nucleic acids that have a higher concentration of smaller nucleic acids that are selectively enriched based on its molecular size. This step may be used to enrich the amount of normally trace nucleic acid, which is initially in the presence of high amounts of non-desired background nucleic acid, to levels suitable for detection and analysis. In some embodiments, CFNAs (e.g., small nucleic acids) are isolated from or enriched in a sample (e.g., a sample prepared or derived from maternal blood or plasma) by one or more following methods: (a) eluting small nucleic acid fragments preferentially from silica, (b) retaining large nucleic acid fragments preferentially on silica, (c) enriching small nucleic acid based on differences in methylation relative to other nucleic acid, (d) enriching small nucleic acid by size exclusion, (e) enriching small nucleic acids by synchronous (or non-synchronous) coefficient of drag alteration sizing, (f) enriching small nucleic acids by solid phase reversible immobilization sizing, (g) enriching small nucleic acids by electrophoresis-based sizing, (h) enriching small nucleic acids by affinity chromatography using iron oxide, i) enriching small nucleic acids by affinity chromatography, and j) enriching small nucleic acids by use of simultaneous anion exchange and size exclusion, wherein the enrichment step involves processing an input sample with one or more of the foregoing techniques produces an output sample comprising a higher concentration of small nucleic acids than the concentration of CFNAs in the input sample. In some embodiments, the enrichment by size exclusion includes using ultrafiltration, size-exclusion chromatography, or dialysis.

Accordingly, in some embodiments, the identification of one or more molecular alterations includes an enrichment step, based on size discrimination, to produce an enriched fraction of cell-free nucleic acids of about 1,000 base pairs or less, about 900 base pairs or less, about 800 base pairs or less, about 700 base pairs or less, about 600 base pairs or less, about 500 base pairs or less, about 400 base pairs or less, about 300 base pairs or less, about 200 base pairs or less, or about 100 base pairs or less. In some embodiments, the enrichment produces an enriched fraction of cell-free nucleic acid of about 1,200 base pairs or less. In some embodiments, the enrichment produces an enriched fraction of cell-free nucleic acid of about 500 base pairs or less. In some embodiments, the enrichment produces an enriched fraction of cell-free nucleic acid of about 200 base pairs or less. In some embodiments, the enrichment produces an enriched fraction of cell-free nucleic acid of about 100 base pairs or less.

In some embodiments, the enrichment includes electrophoresis. Generally, any one of known electrophoretic techniques can be suitable for the methods disclosed herein. In some embodiments, the electrophoresis involved in the enrichment step includes capillary electrophoresis. In some embodiments, the enrichment includes centrifugation. The centrifugation can generally be any one of the centrifugation techniques known in the art can be, for example, micro-centrifugation, high-speed centrifugation, fractional centrifugations, ultra-centrifugations, density gradient centrifugations, and differential centrifugations. In some embodiments, the centrifugation includes gradient centrifugation. In some embodiments, the enrichment includes chromatography. The chromatography can generally be any one of the chromatography techniques known in the art. Suitable centrifugation techniques include, but are not limited to, liquid chromatography, column chromatography, aqueous normal-phase chromatography, size-exclusion chromatography, and two-dimensional chromatography. In some embodiments, the chromatography includes high performance liquid chromatography (HPLC).

In some embodiments of the methods disclosed herein, the one or more molecular alterations associated with MSI is selected from the group consisting of an aberrant production (e.g., elevated production) of the cell-free nucleic acids, a sequence alteration of short tandem DNA repeats, and a fragment-size alteration within short tandem DNA repeats. In some embodiments, the one or more molecular alterations associated with MSI includes a fragment-size alteration within microsatellite short tandem repeats. In some embodiments, the one or more molecular alterations associated with MSI includes aberrant level of cell-free nucleic acids derived from one or more microsatellite loci and/or markers in the patient. In some embodiment, the identification of one or more molecular alterations includes generating a profile of microsatellite loci and/or markers for the patient. Typically, a MSI profile includes a combinations of the following: (1) fragment-size alterations within one or more microsatellite loci and/or markers; (2) sequence alterations within one or more microsatellite loci and/or markers; and (3) expression levels of one or more microsatellite loci and/or markers, which can be derived from patients determined to have or suspected of having a DNA mismatch repair (MMR) deficient cancer. The patient's MSI profile can then be compared to a reference profile. The biomarker profiles, reference and subject, can optionally be contained in a machine-readable medium, such as analog tapes like those readable by a CD-ROM or USB flash media, among others. In addition, the machine-readable media can optionally comprise the subject's other relevant information, e.g., the subject's medical or family history.

In some particular embodiments, the disclosed methods include identifying molecular alterations associated with microsatellite instability (MSI) present in the cell-free nucleic acids, such as, for example sequence alteration and/or a fragment-size alteration within microsatellite short tandem repeats which are characteristic to certain cancers and some other health conditions. In some embodiments, the patient has or is suspected of having a DNA mismatch repair (MMR)-deficient cancer. In some embodiments, the cancer is selected from the group consisting of leukemia, colorectal cancer, breast cancer, lung cancer, prostate cancer, brain tumors, central nervous system tumors, bladder tumors, melanomas, liver cancer, bone cancer, testicular carcinoma, ovarian carcinoma, head and neck tumors, and cervical cancer.

In some embodiments, the methods of the disclosure can include detecting one or more genetic alterations in a target gene known to be associated with microsatellite instability (MSI) in the blood sample. In some embodiments, the target gene is a DNA repair gene. In some embodiments, the DNA repair gene is a gene associated with DNA mismatch repair (MMR), cellular responses to DNA damage, and genetic instability or sensitivity to DNA damaging agents. In some embodiments, the target gene is a gene associated with base excision repair (BER) such as, for example, UNG, SMUG1, MBD4, TDG, OGG1, MUTYH (MYH), NTHL1 (NTH1), MPG, NEIL1, NEIL2, and NEIL3. In some embodiments, the target gene is a gene encoding other BER and strand break joining factors such as, for example, APEX1 (APE1), APEX2, LIG3, XRCC1, PNKP, and APLF (C2ORF13). In some embodiments, the target gene is a gene encoding Poly(ADP-ribose) polymerase (PARP) enzymes that bind to DNA. Non-limiting examples of such enzymes include, but are not limited to, PARP1 (ADPRT), PARP2 (ADPRTL2), and PARP3 (ADPRTL3). In some embodiments, the target gene is a gene associated with the direct reversal of damage or with the repair of DNA-topoisomerase crosslinks, such as, e.g., MGMT, ALKBH2 (ABH2), ALKBH3 (DEPC1), TDP1, and TDP2 (TTRAP). In some embodiments, the target gene is a gene associated with mismatch excision repair and nucleotide excision repair (NER) such as, for example, MSH2, MSH3, MSH6, MLH1, PMS2, MSH4, MSH5, MLH3, PMS1, PMS2L3, XPC, RAD23B, CETN2, RAD23A, XPA, DDB1, DDB2 (XPE), RPA1, RPA2, RPA3, TFIIH, and ERCC3 (XPB). Additional examples of such genes include, but are not limited to, ERCC2 (XPD), GTF2H1, GTF2H2, GTF2H3, GTF2H4, GTF2H5 (TTDA), CDK7, CCNH, MNAT1, ERCC5 (XPG), ERCC1, ERCC4 (XPF), LIG1, ERCC8 (CSA), ERCC6 (CSB), UVSSA (KIAA1530), XAB2 (HCNP), MMS19. Other non-limiting examples of genes of interest include non-homologous end-joining genes such as XRCC6 (Ku70), XRCC5 (Ku80), PRKDC, LIG4, XRCC4, LRE1C (Artemis), and NHEJ1 (XLF, Cernunnos). More information regarding human genes known to be associated with DNA repair can be found in, for example, Wood R D et al., Sciences, Vol. 577, Issues 1-2, 4 Sep. 2005, pp. 275-283; and Wood R D et al., Sciences, Feb. 16 2001; 291(5507):1284-9, which are incorporated herein by reference in their entireties.

In some embodiments, the methods of the disclosure include detecting one or more genetic alterations in at least one target gene selected from the list consisting of SETD1B, RBMXL1, CCDC150, OR7E24, C15orf40, KIAA2018, LTN1, SLC22A9, CDH26, DDX27, EXOSC9, FAM111B, KIAA0182, KIAA1919, MIS18BP1, PRRT2, TMEM60, AQP7, ARV1, CCDC168, ELAVL3, F8, FETUB, HPS1, NBEAL1, P4HTM, PIGB, RBM43, RG9MTD1, SRPR, TMEM97, and any combination thereof. In some embodiments, the disclosed methods include detecting one or more genetic alterations in at least one target gene selected from the list consisting of SETD1B, TMEM60, DDX27, EXOSC9, FAM111B, KIAA1919, and any combination thereof. In some embodiments, the disclosed methods include detecting one or more genetic alterations in at least one target gene selected from the list consisting of SEC31A, CNOT2, RNF145, RNPC3, SLC35F5, TMBIM4, CD3G, DOCKS, MYO10, PRRG1, and any combination thereof.

In some embodiments, the DNA mismatch repair (MMR) gene is selected from the group consisting of Msh2, Msh3, Msh4, Msh5, Msh6, Mlh1, Mlh3, Pms1, Pms2, Exo1, Pol δ, PNCA, RPA, HMGB1, RFC, and DNA ligase I. In some embodiments, the target gene is a gene associated with checkpoint inhibition. In some embodiments, the target gene associated with checkpoint inhibition is selected from the group consisting of PD-1, CTLA-4, A2AR, B7-H3, B7-H4 s, BTLA, IDO, KIR, LAG3, TIM-3, VISTA.

In some embodiments of the disclosure, the at least one checkpoint inhibitor is selected from the group consisting of PD-1 inhibitors, CTLA-4 inhibitors, A2AR inhibitors, B7-H3 inhibitors, B7-H4 inhibitors, BTLA inhibitors, IDO inhibitors, KIR inhibitors, LAG3 inhibitors, TIM-3 inhibitors, VISTA inhibitors, and combinations of any thereof. In some embodiments, the at least one checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C, and combinations thereof.

According to some embodiments of the disclosure, it is envisaged to use more markers than at least two or three. Using more markers will typically yield a more accurate diagnosis although, without being bound to any particular theory, once above a certain threshold of markers, the relative value of adding another marker is limited, as it may not necessarily add information. Thus, according to particular embodiments, at least four, five, six, seven or eight markers are used. According to further particular embodiments, the presence of at least 8, 9, 10, 11 or 12 molecular alterations in microsatellite regions are used to determine the MSI status. According to even further particular embodiments, yet even more markers are used, e.g., at least 15, at least 20, at least 25, at least 30, at least 35, at least 40 markers, or at least 50 markers.

### Methods of Treatment

In one aspect, some embodiments of the disclosure relate to methods for treating a cancer in a patient, including (i) acquiring knowledge of the presence of one or more molecular alterations associated with microsatellite instability (MSI) present in cell-free nucleic acids derived from a blood sample taken from a patient having or being suspected of having a DNA mismatch repair (MMR) deficient cancer; (ii) selecting a therapeutic agent including at least one checkpoint inhibitor appropriate for the treatment of the cancer in the patient based at least in part on whether one or more of the molecular alterations is present in the cell-free nucleic acids; and (iii) administering a therapeutically effective amount of the selected therapeutic agent to the patient.

In another aspect, some embodiments disclosed herein relate to method for treating a patient having cancer, including (1) determining whether a patient has an increased responsiveness for a treatment regime including at least one checkpoint inhibitor by: (i) obtaining cell-free nucleic acids derived from a blood sample taken from a patient having or suspected of having a DNA mismatch repair (MMR) deficient cancer; (ii) identifying one or more molecular alterations associated with microsatellite instability (MSI) present in the cell-free nucleic acids; and (2) administering a therapeutic agent including at least one checkpoint inhibitor appropriate for the treatment of the cancer in the patient if one or more of the molecular alterations is detected in the cell-free nucleic acids.

In yet another aspect, some embodiments disclosed herein relate to a method for treating a patient having cancer, including (1) determining whether a therapeutic agent including at least one checkpoint inhibitor is appropriate for cancer treatment by: (i) obtaining cell-free nucleic acids derived from a blood sample taken from a patient having or suspected of having a DNA mismatch repair (MMR) deficient cancer; (ii) identifying one or more molecular alterations associated with microsatellite instability (MSI) present in the cell-free nucleic acids; and administering a therapeutic agent including at least one checkpoint inhibitor appropriate for the treatment of the cancer in the patient if one or more of the molecular alterations is detected in the cell-free nucleic acids.

In some embodiments disclosed herein, the methods of the disclosure can be used to treat cancer at an early stage of development, cancer that poses few symptoms, cancer that is difficult to distinguish from benign conditions or cancer that can be developing in an area of the body that may not be accessible to traditional biopsy assays.

In some embodiments of the treatment methods disclosed herein, the therapeutic agents described herein (e.g., checkpoint inhibitors and pharmaceutical compositions) can be administered with one or more pharmaceutically-acceptable carriers known in the art using any effective conventional dosage unit forms, including immediate, slow, and timed release preparations. In some embodiments, conventional dosage forms generally provide rapid or immediate drug release from the formulation. In some embodiments, the therapeutic agents described herein (e.g., checkpoint inhibitors and pharmaceutical compositions) can be administered by controlled- or delayed-release means. Controlled-release pharmaceutical products typically have a common goal of improving drug therapy over that achieved by their non-controlled release counterpart.

The phrase “therapeutically effective amount” is an art-recognized term. As used herein, a “therapeutically effective amount” of a therapeutic agent of the disclosure (e.g., an effective dosage) depends on the therapeutic agent selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU) as established by the World Health Organization 1st International Standard for Interleukin-2 (human)). The dosage may be similar to, but is expected to be less than, that prescribed for PROLEUKIN®. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents, checkpoint inhibitors, and pharmaceutical compositions as disclosed herein can include a single treatment or, can include a series of treatments. In one embodiment, the compositions are administered every 8 hours for five days, followed by a rest period of 2 to 14 days, e.g., 9 days, followed by an additional five days of administration every 8 hours.

In some embodiments of the disclosure, the therapeutic agent, e.g., one or more checkpoint inhibitors can be incorporated into compositions, including pharmaceutical compositions. Such compositions typically include the therapeutic agent and a pharmaceutically acceptable carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., therapeutic agents and checkpoint inhibitors as described herein), can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the therapeutic agents as described herein (e.g., checkpoint inhibitors and pharmaceutical compositions) are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the therapeutic agents as described herein (e.g., checkpoint inhibitors and pharmaceutical compositions) can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the therapeutic agents as described herein (e.g., checkpoint inhibitors and pharmaceutical compositions) can also be prepared in the form of suppositories (e.g., with conventional suppository bases such as cocoa butter and other glycerides) or retention enemas for rectal delivery.

In one embodiment, the therapeutic agents as described herein (e.g., checkpoint inhibitors and pharmaceutical compositions) are prepared with carriers that will protect the therapeutic agents, checkpoint inhibitors, and pharmaceutical compositions as disclosed herein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Systems or Kits

In some embodiments of the disclosure, provided herein is a kit for assessing MSI in a CFNA sample, e.g., CFNAs derived from a bodily fluid sample, comprising the tools to genotype the biomarker panel. Other embodiments of the present disclosure relate to detection reagents packaged together in the form of a kit for conducting any of the assays of the present teachings. In certain embodiments, the kits comprise oligonucleotides that specifically identify one or more microsatellites described herein. The oligonucleotide sequences may correspond to fragments of the biomarker nucleic acids. For example, the oligonucleotides can be more than 300, 250, 200, 150, 100, 50, 25, 10, or fewer than 10 nucleotides in length. The kit can contain in separate containers a nucleic acid, control formulations (positive and/or negative), and/or a detectable label, such as but not limited to fluorescein, green fluorescent protein, rhodamine, cyanine dyes, Alexa dyes, luciferase, and radiolabels, among others. Instructions for carrying out the assay, including, optionally, instructions, can be included in the kit.

The kit can contain a nucleic acid substrate array comprising one or more nucleic acid sequences. The nucleic acids on the array specifically identify one or more of the microsatellite regions/loci as described herein. In various embodiments, the sequence (e.g., homopolymer length) of one or more of the microsatellite regions can be identified by virtue of binding to the array. In some embodiments the substrate array can be on a solid substrate, such as what is known as a “chip.” See, e.g., U.S. Pat. No. 5,744,305. In some embodiments the substrate array can be a solution array; e.g., xMAP (Luminex, Austin, Tex.), Cyvera (Illumina, San Diego, Calif.), RayBio Antibody Arrays (RayBiotech, Inc., Norcross, Ga.), CellCard (Vitra Bioscience, Mountain View, Calif.) and Quantum Dots' Mosaic (Invitrogen, Carlsbad, Calif.).

In another aspect, provided herein is a kit that includes one or more of any of the therapeutic agent, checkpoint inhibitors, or pharmaceutical compositions disclosed herein as well as syringes (including pre-filled syringes) and/or catheters (including pre-filled syringes) used to administer any of the therapeutic agent, checkpoint inhibitors, or pharmaceutical compositions to an individual. The kits may also include written instructions for using of any of the therapeutic agents, checkpoint inhibitors, and pharmaceutical compositions as disclosed herein disclosed herein as well as syringes and/or catheters for use with their administration.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

### General Experimental Procedures

The practice of the present disclosure will employ, unless otherwise indicated, conventional techniques of molecular biology, microbiology, cell biology, biochemistry, nucleic acid chemistry, and immunology, which are known to those skilled in the art. Such techniques are explained in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Beaucage et al. eds., Current Protocols in Nucleic Acid Chemistry, John Wiley & Sons, Inc., New York, 2000, (including supplements through 2014), Gene Transfer and. Expression in Mammalian Cells (Makrides, ed., Elsevier Sciences B.V., Amsterdam, 2003), and Current Protocols in Immunology (Horgan K and S. Shaw (1994) (including supplements through 2014). As appropriate, procedures involving the use of commercially available kits and reagents are generally carried out in accordance with manufacturer defined protocols and/or parameters unless otherwise noted.

### Example 2

### Isolation and Quantification of Cell-Free DNA from Colorectal Cancer Patients

Blood samples are collected by venipuncture using vacuum tubes from twenty patients who have been determined to have colorectal cancer. cfDNA from the blood samples are extracted following the same procedure. cfDNA is purified from 200 μL plasma with the QIAamp Circulating Nucleic Acid Kit (Qiagen, CA) according to the manufacturer's recommendations with an elution volume of 60 μL. Samples are kept at 4° C. during plasma preparation. cfDNA samples are frozen at −20° C. until use.

cfDNA is subsequently quantified by Q-PCR assay. Real-time PCR amplifications are carried out in a reaction volume of 25 μL on a My iCycler IQ 5IQ or a Chromo4 instrument using the IQ5 Optical system software 2.0 and the MJ Opticon Monitor 3 software (Bio-Rad). Each PCR reaction mixture consists of 12.5 μL mix PCR (Bio-Rad Super mix SYBR Green-Taq polymerase, MgC1₂); 2.5 μL of each amplification primer (100 pmol/μL); 2.5 μL PCR-analyzed water and 5 μL DNA extract. Melting curves are obtained from 55° C. to 90° C. with reading every 0.2° C. As calibrators for quantification, serial dilutions of genomic DNA from HCT116-S and MC38 cells are used. Sample concentrations are extrapolated from the standard curve by the IQ 5 Optical system software 2.0 or MJ Opticon Monitor 3 software. The limit of detection as the concentration that can be detected with reasonable certainty (95% probability) as recommended in the MIQE guidelines is 3 copies/assay. Typically, in these experiments, no significant difference is found in Q-PCR assay when comparing freshly extracted or stored cfDNA.

### Example 3

### Enrichment of Cell-Free DNA Derived from Colorectal Cancer Patients

In some particular experiments, the cfDNA isolated from colorectal cancer patients as described in Examples 2 are further enriched relative to the cfDNA concentration in the total blood sample, or with respect to the larger cell-bound DNA (e.g., intracellular) fractions. In these instances, the total nucleic acid derived from the blood samples or from the cell-depleted fractions thereof is bound to solid support under appropriate association conditions. Relatively short cfDNA are then purified by collecting the solid support (e.g., by centrifugation or use of a magnetic field for paramagnetic particles), and optionally removing the supernatant to a new tube, after incubating under dissociation conditions for a sufficient period of time that preferentially release the relatively short cfDNA from the solid phase. The relatively short cfDNA is thereby enriched, relative to total nucleic acid by virtue of preferential dissociation from the solid phase relative to the relatively large non-target nucleic acid.

In some particular experiments, additional enrichment of subspecies of relatively short target cfDNA are also performed using similar procedures and the appropriate association and dissociation conditions, using the size selected cfDNA described above. In some particular experiments, further enrichment of subspecies of longer cfDNA are performed by using association conditions sufficient to bind only larger cfDNA, while leaving smaller cfDNA in solution. The solid support is then removed from the non-bound nucleic acids (e.g., smaller cfDNA) in the supernatant, thereby enriching larger nucleic acids.

In some experiments, the enriched nucleic acids are further concentrated. Concentration of nucleic acids is achieved by binding the size selected fraction of nucleic acids, under appropriate association conditions, washing, one or more times, to remove impurities, followed by dissociation (elution) in a smaller volume, of an appropriate buffer or solution, than the original starting volume, thereby concentrating the previously size selected fraction. Alternately, in some particular experiments, concentration of target cfDNA (e.g., having a selected size range) is achieved by precipitating the dissociated target cfDNA.

### Example 4

### Screening of Microsatellite Markers for MSI

In this example, microsatellite markers in cfDNA isolated and enriched from colorectal cancer patients as described in Examples 2-3 are compared to microsatellite markers in cfDNA isolated from healthy individual in order to detect MSI. Specifically, microsatellite loci are amplified from paired healthy/cancer cfDNA samples and genotyped. If one or more different alleles are present in the cancer cfDNA sample that are not found in healthy sample, then it is scored as MSI positive. Di-nucleotide, tetra-nucleotide and pentanucleotide repeat microsatellite markers are then analyzed for frequency of alterations to determine the relative sensitivity of particular markers to MSI. In these experiments, MSI positive samples are divided into three subcategories. The first is characterized by MSI-H (high level of microsatellite instability), wherein a majority of markers exhibit MSI; the second is MSI-L wherein only a minority of markers exhibit MSI; and the third is MSS, wherein markers lack apparent instability. The MSI status of a given cancer patient is determined by looking at microsatellite instability in a panel of genetic markers, comprising two markers which include mononucleotide repeats (BAT26 and BAT25, GenBank Accession Nos. 9834508 and 9834505, respectively), and three markers having dinucleotide repeats (D5S346, D2S123, and D17S250, GenBank Accession Nos 181171, 187953, and 177030, respectively). Using this reference panel, MSI-H tumors are defined as having instability in two or more markers, while MSI-L tumors are defined as having instability in one marker. Tumors showing no apparent instability may be included in the MSS group. In addition to the five primary markers described above, additional markers may be examined for microsatellite instability including, but not limited to, BAT40, BAT34C4, TGF-β-Rll, ACTC(635/636), Dl 8S55, D18S58, D18S61, D18S64, D3S1029, D10S197, D13S175, D17S588, D5S107, D8S87, D18S69, D13S153, D17S787, D7S519, and D20S100. Where more than the primary panel of five markers is used, MSI-H is defined as having MSI in >30-40% of the markers tested, whereas the MSI-L group would exhibit MSI in <30-40% of the markers. A description of the criteria for characterizing MSI-H vs. MSI-L vs. MSS may be found, for example, in Boland et al., 1998, (Cancer Research 58:5248).

The distinction between MSI-H and MSI-L/MSS tumors is important for the prognosis, treatment and monitoring of colorectal cancers. For example, MSI status (that is, the classification of a tumor as MSI-H, MSI-L, or MSS) has been shown to be a predictor of the benefit of adjuvant-based chemotherapy with fluorouracil in stage II and stage III colon cancers (Ribic et al., 2003 N. Engl. J. Med. 349:247). In addition, the proximal anatomical localization of MSI-H tumors has been linked to a more favorable outcome in patients (e.g., higher rate of survival; Boland et al.; Gervaz et al., 2003, Swiss Surg. 9:3; and Rubic et al.), and MSI-H tumors have been correlated to a more favorable stage distribution, with the majority of MSI-H tumors being grouped in Stage II colorectal cancers (Gervaz et al). Thus, the identification and classification of colorectal tumors into MSI status provides the medical practitioner with critical information as to patient diagnosis, prognosis, and optimal treatment regime.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. (canceled)

2. (canceled)

3. A method for selecting a treatment regimen for a patient having a cancer, comprising:
obtaining cell-free nucleic acids obtaining cell-free nucleic acids selected from circulating-free tumor DNAs, circulating-free tumor RNAs, and combinations of any thereof, wherein said cell-free nucleic acids are derived from a blood sample taken from a patient having or suspected of having a DNA mismatch repair (MMR) deficient cancer;
identifying as present in said cell-free nucleic acids one or more molecular alterations associated with microsatellite instability (MSI) selected from the group consisting of an aberrant production of said cell-free nucleic acids, a sequence alteration of short tandem DNA repeats, and a fragment-size alteration within short tandem DNA repeats;
selecting an appropriate treatment regimen for the treatment of said cancer in said patient based at least in part on whether one or more of said molecular alterations is present in said cell-free nucleic acids, wherein said treatment regimen comprises at least one checkpoint inhibitor.

4. (canceled)

5. The method of claim 4, wherein said identifying one or more molecular alterations comprises an analytical assay selected from the group consisting of electrophoresis, chromatography, centrifugation, nucleic acid sequencing, genomic sequencing, next-generation sequencing (NGS), nucleic acid amplification-based assays, nucleic acid hybridization assays, polymerase chain reaction (PCR) assay, real-time PCR assay, quantitative reverse transcription PCR (qRT-PCR) assay.

6. The method of claim 4, wherein said identifying one or more molecular alterations comprises an enrichment process, based on size discrimination, to produce an enriched fraction of cell-free nucleic acids of about 1,200 base pairs or less.

7. The method of claim 5, wherein said enrichment comprises a technique selected from the group consisting of electrophoresis, centrifugation, chromatography, and a combination thereof.

8. The method of claim 7, wherein said electrophoresis comprises capillary electrophoresis.

9. (canceled)

10. The method of claim 7, wherein said centrifugation comprises gradient centrifugation.

11. (canceled)

12. The method of claim 7, wherein said chromatography comprises high performance liquid chromatography (HPLC).

13. The method of claim 6, wherein said enrichment produces an enriched fraction of cell-free nucleic acid of about 500 base pairs or less.

14-17. (canceled)

18. The method of claim 13, further comprising detecting one or more genetic alterations in a target gene known to be associated with microsatellite instability (MSI) in said blood sample.

19. (canceled)

20. (canceled)

21. The method of claim 18, wherein said target gene is a gene associated with checkpoint inhibition selected from the group consisting of PD-1, CTLA-4, A2AR, B7-H3, B7-H4, BTLA, IDO, KIR, LAG3, TIM-3, and VISTA.

22. (canceled)

23. (canceled)

24. The method of claim 21, wherein said detecting one or more genetic alterations comprises an analytical assay selected from the group consisting of electrophoresis, nucleic acid sequencing, nucleic acid amplification-based assays, nucleic acid hybridization assays, polymerase chain reaction (PCR) assay, real-time PCR assay, quantitative reverse transcription PCR (qRT-PCR) assay, genomic sequencing, next-generation sequencing (NGS).

25-29. (canceled)

30. A method for treating a cancer in a patient, comprising:
acquiring knowledge of the presence of one or more molecular alterations associated with microsatellite instability (MSI) present in cell-free nucleic acids derived from a blood sample taken from a patient having or being suspected of having a DNA mismatch repair (MMR) deficient cancer;
selecting a therapeutic agent comprising at least one checkpoint inhibitor appropriate for the treatment of said cancer in said patient based at least in part on whether one or more of said molecular alterations is present in said cell-free nucleic acids; and
administering a therapeutically effective amount of said selected therapeutic agent to said patient.

31. (canceled)

32. A method for treating a cancer patient, comprising determining whether a therapeutic agent comprising at least one checkpoint inhibitor is appropriate for cancer treatment by:
obtaining cell-free nucleic acids selected from circulating-free tumor DNAs, circulating-free tumor RNAs, and combinations of any thereof, wherein said cell-free nucleic acids are derived from a blood sample taken from a patient having or suspected of having a DNA mismatch repair (MMR) deficient cancer;
identifying as present in said cell free nucleic acids one or more molecular alterations associated with microsatellite instability (MSI) selected from the group consisting of an aberrant production of said cell-free nucleic acids, a sequence alteration of short tandem DNA repeats, and a fragment-size alteration within short tandem DNA repeats; and
administering a therapeutic agent comprising at least one checkpoint inhibitor appropriate for the treatment of said cancer in said patient if one or more of said molecular alterations is detected in said cell-free nucleic acids.

33. (canceled)

34. The method of claim 33, wherein said identifying one or more molecular alterations comprises an analytical assay selected from the group consisting of electrophoresis, chromatography, centrifugation, nucleic acid sequencing, genomic sequencing, next-generation sequencing (NGS), nucleic acid amplification-based assays, nucleic acid hybridization assays, polymerase chain reaction (PCR) assay, real-time PCR assay, quantitative reverse transcription PCR (qRT-PCR) assay.

35. The method of claim 33, wherein said identifying one or more molecular alterations comprises an enrichment step, based on size discrimination, to produce an enriched fraction of cell-free nucleic acids of about 1,200 base pairs or less.

36. The method of claim 35, wherein said enrichment comprises a technique selected from the group consisting of electrophoresis, centrifugation, chromatography, and a combination thereof.

37. The method of claim 36, wherein said electrophoresis comprises capillary electrophoresis.

38. (canceled)

39. The method of claim 36, wherein said centrifugation comprises gradient centrifugation.

40. (canceled)

41. The method of claim 36, wherein said chromatography comprises high performance liquid chromatography (HPLC).

42. The method of claim 35, wherein said enrichment produces an enriched fraction of cell-free nucleic acid of about 500 base pairs or less.

43-60. (canceled)
